(19) United States
(12) Patent Application Publication
(10) Pub. No.: US 2016/0354060 A1
(43) Pub. Date: Dec. 8, 2016

(54) METHODS AND SYSTEMS FOR CONTROLLING A DIAGNOSTIC MEDICAL IMAGING DISPLAY INTERFACE

(71) Applicant: General Electric Company, Schenectady, NY (US)

(72) Inventors: CHRISTIAN FRITZ PERREY, Zipf (AT); Peter Falkensammer, Zipf (AT); Martin Paul Mienkina, Zipf (AT); Barbara Kari, Wauwatosa, WI (US)

(21) Appl. No.: 14/728,352

(22) Filed: Jun. 2, 2015

(51) Int. Cl. A61B 8/00; A61B 8/08

(52) U.S. Cl. CPC A61B 8/465; A61B 8/5207

(57) ABSTRACT

Methods and systems for a display interface are provided. The systems and methods receive ultrasound image acquisition settings for an ultrasound probe, and acquire ultrasound image data from the ultrasound probe based on the ultrasound image acquisition settings. The systems and methods determine one or more image characteristics of the ultrasound image data, and adjust one or more image display settings corresponding to a first interface component based on the one or more image characteristics. Further, the systems and methods display on a display the interface component and may additionally display the ultrasound image.

### BACKGROUND OF THE INVENTION

Embodiments described herein generally relate to display interfaces for diagnostic medical imaging, and more particularly to a controlling display interface for an ultrasound system.

Diagnostic medical imaging systems typically include a scan portion and a control portion having a display. For example, ultrasound imaging systems usually include ultrasound scanning devices, such as ultrasound probes having transducers that are connected to an ultrasound system to control the acquisition of ultrasound data during an examination (e.g., ultrasound scan) by a user to acquire one or more ultrasound images or videos (e.g., imaging the volume or body) of a patient.

During the examination, the user may select, activate, or switch repeatedly between different components displayed on a display. For example, the components may include patient report information, interface items such as pop-up windows or drop-down menus, and/or the like. The components on the display have predetermined default image display settings such as a different hue, brightness, contrast, or saturation settings. However, the display settings for display components are separate and distinct from the displays settings of the ultrasound images. For example, acquired ultrasound images or videos have image display settings that are based on the acquisition settings of the ultrasound probe. Consequently, the acquired ultrasound images or videos are displayed with display settings that constantly change and differ from the display settings of the display components. Exposure to repeated changes of the image display settings overtime causes eye fatigue for the user. For example, during one examination, the acquired ultrasound images may be darker relative to a drop-down menu and other components displayed on the display, while during another examination, the ultrasound images may be lighten relative to the menus and other components.

Conventional ultrasound image systems allow the user to manually adjust the display settings of the components. However, these adjustments are cumbersome and are applied to all users of the system. For these and other reasons, an improved display interface is needed for diagnostic medical imaging.

### BRIEF DESCRIPTION OF THE INVENTION

In one embodiment, a method for controlling an ultrasound interface is provided. The method may include receiving ultrasound image acquisition settings for an ultrasound probe, and acquiring ultrasound image data from the ultrasound probe based on the ultrasound image acquisition settings. The method may also include determining one or more image characteristics of the ultrasound image data, and adjusting one or more image display settings corresponding to a first interface component based on the one or more image characteristics. Further, the method may include displaying on a display an ultrasound image concurrently with the first interface component. The first interface component displayed based on the one or more images display settings from the adjusting operation. The ultrasound image is based on the ultrasound image data.

In another embodiment, an ultrasound imaging system is provided. The ultrasound imaging system may include an ultrasound probe configured to acquire ultrasound data from a region of interest, and a display. The ultrasound imaging system may also include a controller circuit that includes at least one processor operably coupled to the ultrasound probe and the display. The controller circuit may be configured to receive ultrasound image data from the ultrasound probe, determine one or more image characteristics of the ultrasound image data, and adjust one or more image display settings corresponding to a first interface component based on the one or more image characteristics. The controller circuit may also be configured to generate a display signal for the display corresponding to an ultrasound image shown concurrently with the first interface component. The first interface component displayed based on the one or more image display settings from the adjusting operation. The ultrasound image is based on the ultrasound image data.

In another embodiment, a tangible and non-transitory computer readable medium may include one or more computer software modules configured to direct one or more processors. The one or more computer software modules may be configured to direct the one or more processors to receive medical image data, determine one or more image characteristics of the ultrasound image data, and adjust one or more image display settings corresponding to a first interface component based on the one or more image characteristics. The one or more computer software modules may also be configured to direct the one or more processors to generate a display signal for the display corresponding to an ultrasound image shown concurrently with the first interface component. The first interface component displayed based on the one or more image display settings from the adjusting operation. The ultrasound image is based on the ultrasound image data.

### BRIEF DESCRIPTION OF THE DRAWINGS

FIG. 1 illustrates a schematic block diagram of an ultrasound imaging system, in accordance with an embodiment.

FIG. 2 illustrates a display interface shown on a display of an ultrasound imaging system, in accordance with an embodiment.

FIG. 3 illustrates a flowchart of a method for displaying an ultrasound interface, in accordance with an embodiment.

FIG. 4 is an illustration of a simplified block diagram of a controller circuit of the ultrasound imaging system of FIG. 1, in accordance with an embodiment.

FIG. 5 is a graphical illustration of a characterization histogram corresponding to an image characteristic of an ultrasound image, in accordance with an embodiment.

FIG. 6 is a graphical illustration of a characterization histogram corresponding to an interface component of a display interface, in accordance with an embodiment.

FIG. 7 illustrates a 3D capable miniaturized ultrasound system having a probe that may be configured to acquire 3D ultrasonic data or multi-plane ultrasonic data.

FIG. 8 illustrates a hand carried or pocket-sized ultrasound imaging system wherein the display and user interface form a single unit.

FIG. 9 illustrates an ultrasound imaging system provided on a movable base.

### DETAILED DESCRIPTION OF THE INVENTION

The following detailed description of certain embodiments will be better understood when read in conjunction with the appended drawings. To the extent that the figures illustrate diagrams of the functional modules of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or a block of random access memory, hard disk, or the like). Similarly, the programs may be stand-alone programs, may be incorporated as subroutines in an operating system, may be functions in an installed software package, and the like. It should be understood that the various embodiments are not limited to the arrangements and instrumentality shown in the drawings.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising” or “having” an element or a plurality of elements having a particular property may include additional elements not having that property.

Various embodiments provide systems and methods for display interfaces for diagnostic medical imaging, and more particularly for an ultrasound interface that links the image display settings of one or more interface components (e.g., menus, pop-up windows) shown on the display to the image display settings of one or more ultrasound images selected by a user (e.g., clinician, doctor, sonographer). Prior to acquisition of the medical image data, the user may adjust the acquisition settings based on the region of interest (ROI) and/or type of examination being performed. For example, the user may select and/or adjust gain, power, time gain compensation, color map presets, and/or the like for an ultrasound probe prior to acquisition of ultrasound data or data. Based on the acquisition settings, one or more medical images are acquired with corresponding image display settings (e.g., hue, brightness, contrast, saturation).

Based on the image display settings of the one or more medical images, various embodiments may adjust one or more interface components of the user interface (e.g., menus, drop-down menus, pop-up windows, selectable icons, a toolbar, a menu bar, a title bar, a window). For example, a mean brightness and contrast of the one or more medical images is calculated by one or more processors. A contrast and brightness of the one or more interface components are adjusted to match the calculated mean brightness and contrast of the one or more medical images.

Additionally or alternatively, when the user changes to other display screens via selection of the user interface, one or more processors may continually adjust one or more interface components of the other display screens to match the calculated mean brightness and contrast of the one or more medical images.

Optionally, the adjustment to the one or more interface components may be based on the content of the one or more interface components. For example, a contrast of a text drop-down menu may be optimized based on the calculated mean brightness value of the one or more medical images and not the mean contrast.

A technical effect of at least one embodiment includes reduced eye fatigue of users relative to conventional diagnostic medical imaging systems. A technical effect of at least one embodiment includes a reduction in different image display settings between medical exams (e.g., ultrasound exams) and other menus of the user interface.

It should be noted that although the various embodiments may be described in connection with an ultrasound system, the methods and systems are not limited to ultrasound imaging or a particular configuration thereof. The various embodiments may be implemented in connection with different types of diagnostic medical imaging systems, including, for example, x-ray imaging systems, magnetic resonance imaging (MRI) systems, computed-tomography (CT) imaging systems, positron emission tomography (PET) imaging systems, or combined imaging systems, among others.

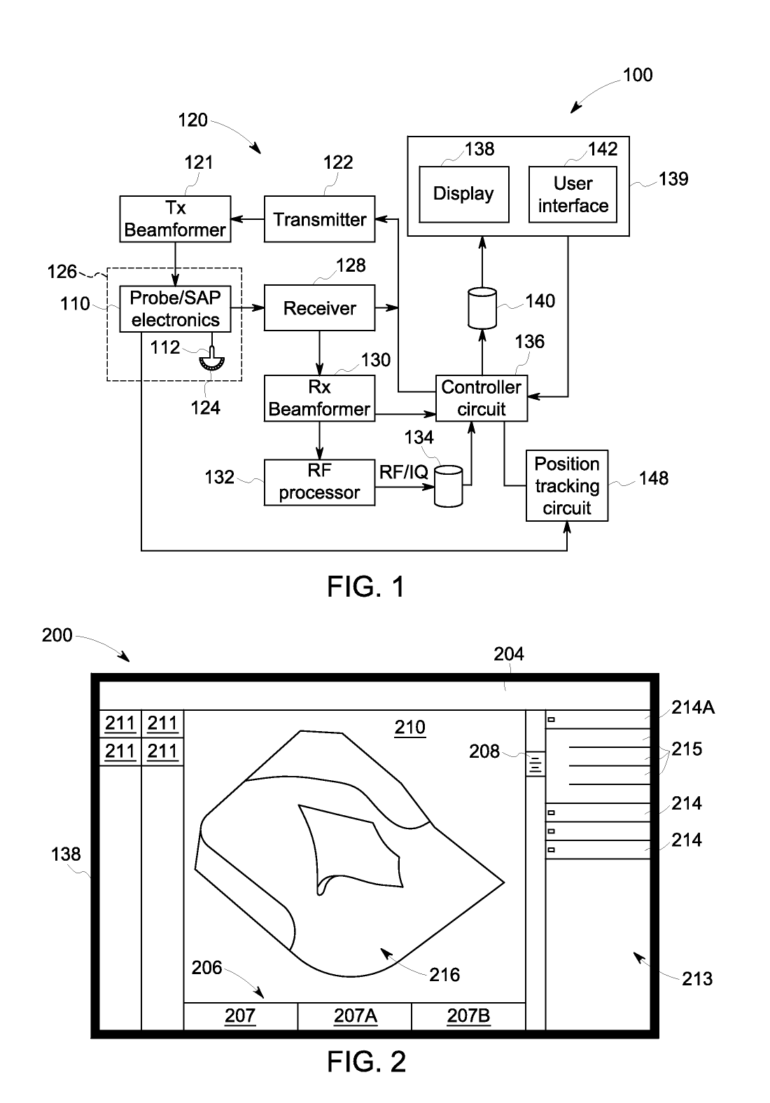

FIG. 1 is a schematic diagram of a diagnostic medical imaging system, specifically, an ultrasound imaging system 100. The ultrasound imaging system 100 includes an ultrasound probe 126 having a transmitter 122 and probe/SAP electronics 110. The ultrasound probe 126 may be configured to acquire ultrasound data or information from a region of interest (e.g., organ, blood vessel) of the patient. The ultrasound probe 126 is communicatively coupled to the controller circuit 136 via the transmitter 122. The transmitter 122 transmits a signal to a transmit beamformer 121 based on acquisition settings received by the user. The signal transmitted by the transmitter 122 in turn drives the transducer elements 124 within the transducer array 112. The transducer elements 124 emit pulsed ultrasonic signals into a patient (e.g., a body). A variety of a geometries and configurations may be used for the array 112. Further, the array 112 of transducer elements 124 may be provided as part of, for example, different types of ultrasound probes.

The acquisition settings may define an amplitude, pulse width, frequency, and/or the like of the ultrasonic pulses emitted by the transducer elements 124. The acquisition settings may be adjusted by the user by selecting a gain setting, power, time gain compensation (TGC), resolution, and/or the like from the user interface 142.

The transducer elements 124, for example piezoelectric crystals, emit pulsed ultrasonic signals into a body (e.g., patient) or volume corresponding to the acquisition settings. The ultrasonic signals may include, for example, one or more reference pulses, one or more pushing pulses (e.g., shear-waves), and/or one or more tracking pulses. At least a portion of the pulsed ultrasonic signals back-scatter from a region of interest (ROI) (e.g., breast tissues, liver tissues, cardiac tissues, prostate tissues, and the like) to produce echoes. The echoes are delayed in time according to a depth, and are received by the transducer elements 124 within the transducer array 112. The ultrasonic signals may be used for imaging, for generating and/or tracking shear-waves, for measuring differences in compression displacement of the tissue (e.g., strain), and/or for therapy, among other uses. For example, the probe 126 may deliver low energy pulses during imaging and tracking, medium to high energy pulses to generate shear-waves, and high energy pulses during therapy.

The transducer array 112 may have a variety of array geometries and configurations for the transducer elements 124 which may be provided as part of, for example, different types of ultrasound probes 126. The probe/SAP electronics 110 may be used to control the switching of the transducer elements 124. The probe/SAP electronics 110 may also be used to group the transducer elements 124 into one or more sub-apertures.

The transducer elements 124 convert the received echo signals into electrical signals which may be received by a receiver 128. The electrical signals representing the received echoes are passed through a receive beamformer 130, which performs beamforming on the received echoes and outputs a radio frequency (RF) signal. The RF signal is then provided to an RF processor 132 that processes the RF signal. The RF processor 132 may generate different ultrasound image data types, e.g. B-mode, color Doppler (velocity/power/variance), tissue Doppler (velocity), and Doppler energy, for multiple scan planes or different scanning patterns. For example, the RF processor 132 may generate tissue Doppler data for multi-scan planes. The RF processor 132 gathers the information (e.g. I/Q, B-mode, color Doppler, tissue Doppler, and Doppler energy information) related to multiple data slices and stores the data information, which may include time stamp and orientation/rotation information, on the memory 134.

Alternatively, the RF processor 132 may include a complex demodulator (not shown) that demodulates the RF signal to form IQ data pairs representative of the echo signals. The RF or IQ signal data may then be provided directly to a memory 134 for storage (e.g., temporary storage). Optionally, the output of the beamformer 130 may be passed directly to a controller circuit 136.

The controller circuit 136 may be configured to process the acquired ultrasound data (e.g., RF signal data or IQ data pairs) and prepare frames of ultrasound image data for display on the display 138. The controller circuit 136 may include one or more processors. Optionally, the controller circuit 136 may include a central controller circuit (CPU), one or more microprocessors, a graphics controller circuit (GPU), or any other electronic component capable of processing inputted data according to specific logical instructions. Having the controller circuit 136 that includes a GPU may be advantageous for computation-intensive operations, such as volume-rendering. Additionally or alternatively, the controller circuit 136 may execute instructions stored on a tangible and non-transitory computer readable medium (e.g., the memory 140).

The controller circuit 136 is configured to perform one or more processing operations according to a plurality of selectable ultrasound modalities on the acquired ultrasound data, adjust or define the ultrasonic pulses emitted from the transducer elements 124, adjust one or more image display settings of components (e.g., ultrasound images, interface components) displayed on the display 138, and other operations as described herein. Acquired ultrasound data may be processed in real-time by the controller circuit 136 during a scanning or therapy session as the echo signals are received. Additionally or alternatively, the ultrasound data may be stored temporarily on the memory 134 during a scanning session and processed in less than real-time in a live or off-line operation.

The ultrasound imaging system 100 may include a memory 140 for storing processed frames of acquired ultrasound data that are not scheduled to be displayed immediately or to store post-processed images (e.g., shear-wave images, strain images), firmware or software corresponding to, for example, a graphical user interface, one or more default image display settings, and/or the like. The memory device 140 may be a tangible and non-transitory computer readable medium such as flash memory, RAM, ROM, EEPROM, and/or the like.

One or both of the memory 134 and 140 may store 3D ultrasound image data sets of the ultrasound data, where such 3D ultrasound image data sets are accessed to present 2D and 3D images. For example, a 3D ultrasound image data set may be mapped into the corresponding memory 134 or 140, as well as one or more reference planes. The processing of the ultrasound data, including the ultrasound image data sets, may be based in part on user inputs, for example, user selections received at the user interface 142.

The ultrasound imaging system 100 may include a position tracking circuit 148. The position tracking circuit 148 tracks a position of the probe 126 and communicates the position to the controller circuit 136 as described above.

The controller circuit 136 is operably coupled to a display 138 and a user interface 142. The display 138 may include one or more liquid crystal displays (e.g., light emitting diode (LED) backlight), organic light emitting diode (OLED) displays, plasma displays, CRT displays, and/or the like. The display 138 may display patient information, ultrasound images and/or videos, components of a display interface, one or more 2D, 3D, or 4D ultrasound image data sets from ultrasound data stored on the memory 134 or 140 or currently being acquired, measurements, diagnosis, treatment information, and/or the like received by the display 138 from the controller circuit 136.

The user interface 142 controls operations of the controller circuit 136 and is configured to receive inputs from the user. The user interface 142 may include a keyboard, a mouse, a touchpad, one or more physical buttons, and/or the like. Optionally, the display 138 may be a touch screen display, which includes at least a portion of the user interface 142. For example, the user may select one or more user selectable elements shown on the display by touching or making contact with the display 138.

FIG. 2 illustrates a display interface 200 shown on the display 138, in accordance with an embodiment. The display interface 200 may correspond to a graphical user interface (GUI) generated by the controller circuit 136. The display interface 200 may include one or more interface components and/or an activity window 210. The activity window 210 may correspond to an area of the display interface 200 for viewing results of one or more operations performed by the controller circuit 136. For example, the activity window 210 may include one or more ultrasound images 216, ultrasound videos, measurements, diagnostic results, data entry (e.g., patient information), and/or the like. It should be noted in various other embodiments the activity window 210 may be larger or smaller relative to the one or more interface components illustrated in the display interface 200. Optionally the activity window 210 may be in a full-screen mode. For example, a size of the activity window 210 may encompass the display interface 200 (e.g., no interface components are included in the display interface 200).

The interface components correspond to user selectable elements shown visually on the display 138, and may be selected, manipulated, and/or activated by the user operating the user interface 142 (e.g., touch screen, keyboard, mouse). The interface components may be presented in varying shapes and colors, such as a graphical or selectable icon 211, slide bar 208, a cursor, and/or the like. Optionally, one or more interface components may include text or symbols, such as a drop-down menu 214, a toolbar 206, a menu bar 213, a title bar 204, a window (e.g., a pop-up window) and/or the like. Additionally or alternatively, one or more interface components may indicate areas within the display interface 200 for entering or editing information (e.g., patient information, user information, diagnostic information) within the display interface 200, such as a text box, a text field, and/or the like.

The menu bar 213 may correspond to a list of textual or graphical user selectable elements from which the user may select. For example, the menu bar 213 may include one or more drop-down menus 214 that correspond to one or more operations or functions that may be performed by the controller circuit 136 when selected by the user.

The toolbar 206 may correspond to an area of the display interface 200 that is subdivided into tabs or selectable icons 207 corresponding to select operation modes of the ultrasound imaging system 100. For example, the selectable icon 207a may correspond to a patient entry/access mode. When selected by the user, the controller circuit 136 may display one or more interface components relating to selecting and/or editing patient records, viewing a patient history list, entering new patient information, and/or the like. In another example, the selectable icon 207b may correspond to an imaging mode. When selected by the user, the controller circuit 136 may display one or more interface components relating to acquiring ultrasound image data, performing diagnostic on ultrasound images, setting acquisition settings for the ultrasound probe 126, and/or the like. In various embodiments, the toolbar 206 and/or selectable icons 207 may be static, for example, remaining in the same position relative to the display interface 200 for two more selected operation modes.

The title bar 204 may identify information of the patient, user information, data and/or time information, and/or the like during operation of the ultrasound imaging system 100.

The slide bar 208 may allow the user to adjust a viewable area of one or more interface components or the activity window 210 to make a different portion of the corresponding one or more interface components or the activity window 210 viewable. For example, not all of the drop-down menus 214 can be viewed within the menu bar 213. The user may adjust a position of the slide bar 208 to replace one or more of the currently viewable drop-down menus with the hidden one or more drop-down menus.

It should be noted various other embodiments may include additional or fewer interface components, differently sized interface components, and/or interface components having a different orientation or position relative to the interface components shown in FIG. 2.

In various embodiments, the interface components may perform various functions when selected, such as measurement functions, editing functions, database access/search functions, diagnostic functions, controlling acquisition settings, and/or system settings for the ultrasound imaging system 100 performed by the controller circuit 136. For example, the drop-down menu 214a may correspond to a category of visual diagnostic selections generated by the controller circuit 136 that may be selected by the user. When the user selects the drop-down menu 214a using the user interface 142, one or more submenu components 215 may be displayed. Each submenu component 215 may correspond to a visual diagnostic (e.g., color flow, B-mode, electrography, spectral Doppler). When the user selects one of the submenu components 215 using the user interface 142, the controller circuit 136 may receive the request and generate and/or perform the selected operation.

The one or more interface components may have corresponding image display settings. The image display settings may be used by the controller circuit 136 to render or generate the one or more interface components to be displayed by the display 138. For example, the image display settings may define the color, size, and/or brightness of one or more pixels forming each of the interface components shown on the display interface 200. Optionally, the image display settings may include shape and/or position information of the one or more interface components. The image display settings may be stored on an image display settings table or database stored on the memory 140.

For example, the image display settings database may include a plurality of interface components with a corresponding image display setting(s) of the interface component. Each of the interface components may have a unique image display setting. The image display settings database may be used by the controller circuit 136 to render or generate the corresponding interface component. For example, the user may activate the drop-down menu 214a instructing the controller circuit 136 to generate the submenu components 215 on the display 138. The controller circuit 136 may locate the appropriate submenu components 215 on the image display settings database stored on the memory 140, and generate the submenu components 215 using the corresponding image display settings from the image display settings database.

In various embodiments, the image display settings may further include the luminance (e.g., brightness, relative luminance) and chromaticity values of the red green, and blue primaries (e.g., the color) of the one or more pixels forming each interface component. The chromaticity values may correspond to a hue and a saturation (e.g., colorfulness, chroma, intensity) of the one or more pixels. Based on the image display settings, the controller circuit 136 may generate a display signal, which is received by the display 138.

The display signal may be a video interface (e.g., Video Graphics Array, DisplayPort, High Definition Multimedia Interface, Digital Visual Interface, MHL, SDI, and/or the like) which is used by the display 138. The display signal may correspond to a series of pixel configurations from the controller circuit 136, and used by the display 138 for displaying the display interface 200 (e.g., the ultrasound image 216 shown concurrently with one or more interface components). For example, the display signal may be a series of packets along three channels corresponding to a red, green, and blue intensity value, respectively, of a pixel. The display 138 may adjust red, green, and blue intensity values of the pixels based on the received display signal.

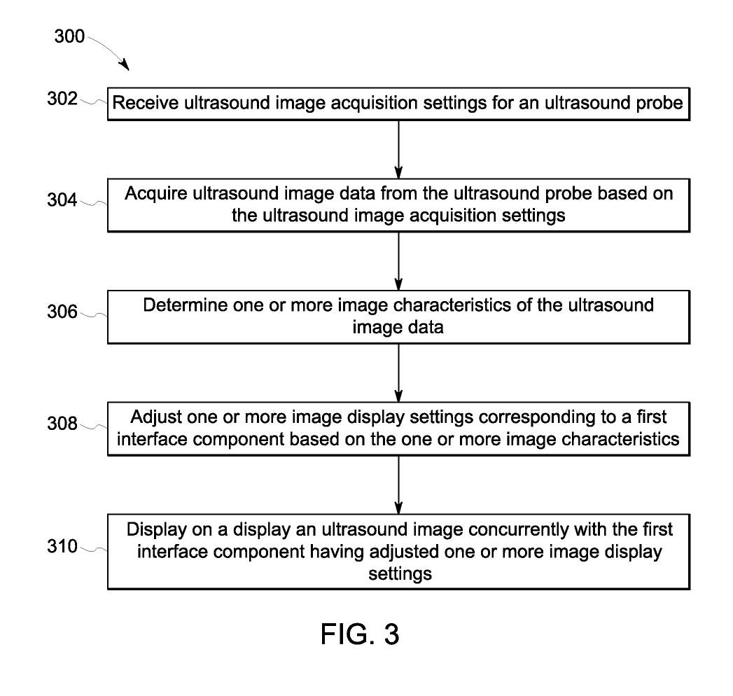

In connection with FIG. 3, the controller circuit 136 may adjust one or more image display settings of one or more interface components based on the ultrasound data, such as the ultrasound image data, acquired by the ultrasound probe 126. Adjusting the one or more image display settings may effect a brightness, a contrast, a color, a hue, saturation, and/or the like of one or more interface components of the display interface 200 shown on the display 138.

FIG. 3 illustrates a flowchart of a method 300 for displaying an ultrasound interface, in accordance with various embodiments described herein. The method 300, for example, may employ structures or aspects of various embodiments (e.g., systems and/or methods) discussed herein. In various embodiments, certain steps (or operations) may be omitted or added, certain steps may be combined, certain steps may be performed simultaneously, certain steps may be performed concurrently, certain steps may be split into multiple steps, certain steps may be performed in a different order, or certain steps or series of steps may be re-performed in an iterative fashion. In various embodiments, portions, aspects, and/or variations of the method 300 may be used as one or more algorithms to direct hardware to perform one or more operations described herein. It should be noted, other methods may be used, in accordance with embodiments herein.

One or more methods may (i) receive ultrasound image acquisition settings for an ultrasound probe; (ii) acquire ultrasound image data from the ultrasound probe based on the ultrasound image acquisition settings; (iii) determine one or more image characteristics of the ultrasound image data; (iv) adjust one or more image display settings corresponding to a first interface component based on the one or more image characteristics; and (v) display on a display an ultrasound image concurrently with the first interface component having adjusted one or more image display settings.

Beginning at 302, ultrasound acquisition settings are received for the ultrasound probe 126. The ultrasound acquisition settings may be selected from the user via the user interface 142 (FIG. 1). For example, the user may define the gain, power, time gain compensation (TGC), resolution, and/or the like of the probe using the user interface 142. Optionally, the ultrasound acquisition settings may be received by the controller circuit 136 from selections of the user of one or more interface components (e.g., selectable icons 211, drop-down menus 214) of the display interface 200 (FIG. 2) shown on the display 138.

Optionally, the acquisition settings may be selected to generate an ultrasound image having select one or more image characteristics. The one or more image characteristics may correspond to a color (e.g., hue, saturation), brightness (e.g., luminosity), contrast, and/or the like of the ultrasound image. For example, to increase the brightness of an ultrasound image the TGC may be increased. In another example, to decrease the amount of contrast and/or saturation of an ultrasound image the gain may be decreased.

At 304, ultrasound image data is acquired from the ultrasound probe 126 based on the ultrasound image acquisition settings. In reference to FIG. 1, the acquisition settings may define an amplitude, pulse width, frequency, and/or the like of the ultrasonic pulses emitted by the transducer elements 124. The transducer elements 124 of the ultrasound probe 126 emit the ultrasonic pulses into a region of interest (ROI) of the patient, of which, at least a portion of the pulsed ultrasonic signals back-scatter from the ROI to produce echoes and are received by the transducer elements 124. The transducer elements 124 convert the received echo signals into electrical signals which may be received by a receiver 128. The electrical signals representing the received echoes are passed through the receive beamformer 130 and is then provided to the RF processor 132. The RF processor 132 may generate the ultrasound image data the ultrasound data, which may then be stored on the memory 134 for storage (e.g., temporary storage). Additionally or alternatively, the output of the receive beamformer 130 may be passed directly to the controller circuit 136.

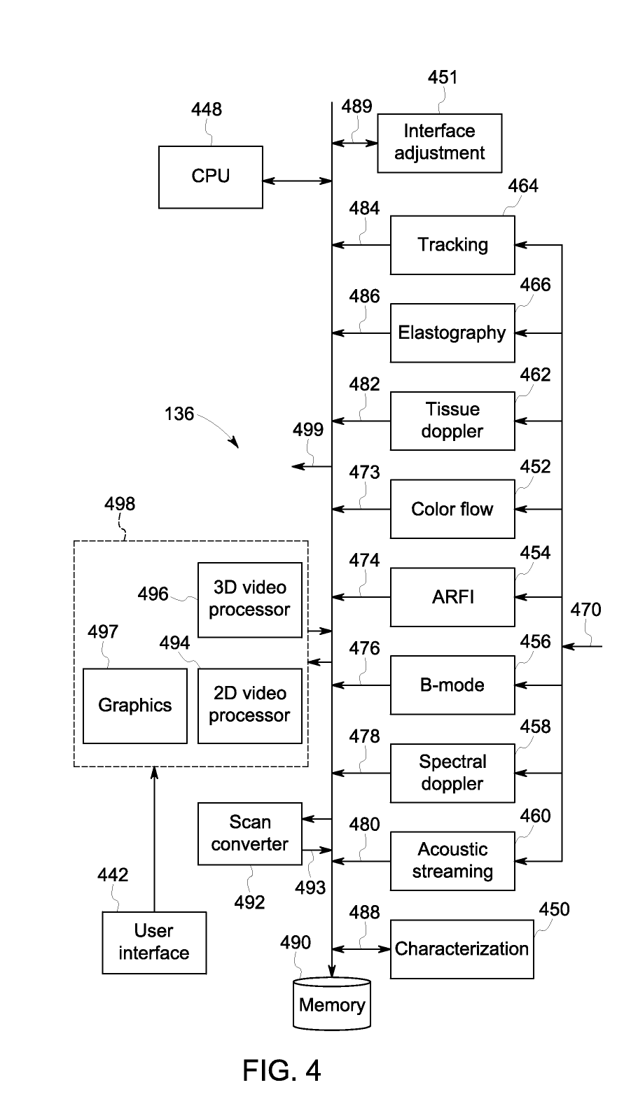

Additionally or alternatively, in connection with FIG. 4, the ultrasound image data may further be determined by the controller circuit 136.

FIG. 4 is an exemplary block diagram of the controller circuit 136. The controller circuit 136 is illustrated in FIG. 4 conceptually as a collection of circuits and/or software modules, but may be implemented utilizing any combination of dedicated hardware boards, DSPs, one or more processors, FPGAs, ASICs, a tangible and non-transitory computer readable medium configured to direct one or more processors, and/or the like.

The circuits 450-466 (e.g., dedicated hardware, micro-processors, software modules) perform mid-processor operations representing one or more visual diagnostics, operations, data manipulation, and/or the like of the ultrasound imaging system 100. The circuits 450-466 may be controlled by a local central processing unit 448. The controller circuit 136 may receive ultrasound data 470 in one of several forms. In the embodiment of FIG. 4, the received ultrasound data 470 constitutes IQ data pairs representing the real and imaginary components associated with each data sample. The IQ data pairs are provided to one or more circuits, for example, a color-flow circuit 452, an acoustic radiation force imaging (ARFI) circuit 454, a B-mode circuit 456, a spectral Doppler circuit 458, an acoustic streaming circuit 460, a tissue Doppler circuit 462, a tracking circuit 464, and an elastography circuit 466. Other circuits may be included, such as an M-mode circuit, power Doppler circuit, among others. However, embodiments described herein are not limited to processing IQ data pairs. For example, processing may be done with RF data and/or using other methods. Furthermore, data may be processed through multiple circuits.

Each of circuits 452-466 is configured to process the IQ data pairs in a corresponding manner to generate, respectively, color-flow data 473, ARFI data 474, B-mode data 476, spectral Doppler data 978, acoustic streaming data 480, tissue Doppler data 482, tracking data 484 (e.g., ROI data acquisition location), elastography data 486 (e.g., strain data, shear-wave data), among others, all of which may be stored in a memory 490 (or memory 134 or memory 140 shown in FIG. 1) temporarily before subsequent processing. The data 473-486 may be stored, for example, as sets of vector data values, where each set defines an individual ultrasound image frame. The vector data values are generally organized based on the polar coordinate system. The memory device 490 may be a tangible and non-transitory computer readable medium such as flash memory, RAM, ROM, EEPROM, and/or the like.

A scan converter circuit 492 accesses and obtains from the memory 490, the vector data values associated with an image frame and converts the set of vector data values to Cartesian coordinates to generate an ultrasound image frame 493 formatted for display. The ultrasound image frames 493 generated by the scan converter circuit 492 may be provided back to the memory 490 for subsequent processing or may be provided to the memory 434 or the memory 440. Once the scan converter circuit 492 generates the ultrasound image frames 493 associated with the data, the ultrasound image frames 493 may be stored on the memory 490 or communicated over a bus 499 to a database (not shown), the memory 134, the memory 140, and/or to other processors (not shown).

Returning to FIG. 3, at 306, one or more image characteristics of the ultrasound image data is determined. The one or more image characteristics may correspond to a color (e.g., hue, saturation), brightness (e.g., luminosity), contrast, and/or the like of an ultrasound image, such as from one or more ultrasound image frames 493 stored on the memory 490. The one or more image characteristics may be determined by the characterization circuit 450 of the controller circuit 136.

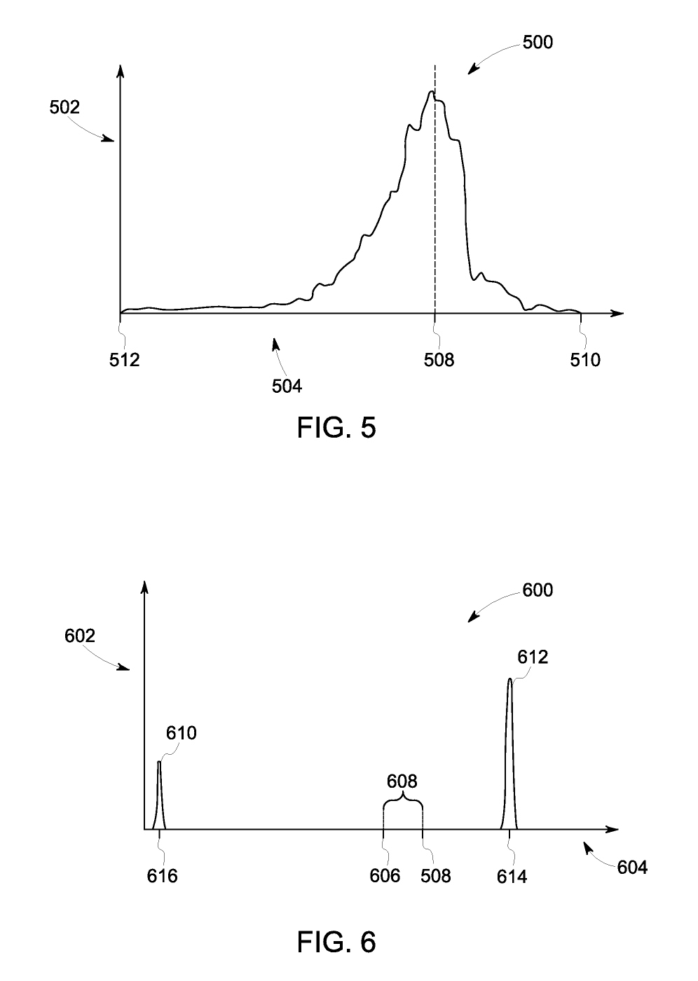

For example, the characterization circuit 450 may retrieve or access select ultrasound image data 488 (e.g., the ultrasound image 216 of FIG. 2, one of the ultrasound image frames 493) from the memory 490. The characterization circuit 450 may calculate an average (e.g., geometric mean, arithmetic mean, mode, median) of the one or more image characteristics of the ultrasound image data 488, which may be stored on the memory 490. The characterization circuit 450 may determine from each pixel of the select ultrasound image data 488 one or more image characteristics, such as brightness, which may be viewed as a histogram as shown in FIG. 5.

FIG. 5 is a graphical representation of a characterization histogram 500 corresponding to the brightness of the pixels of the ultrasound image data 488. The brightness of the pixels may have been determined by the characterization circuit 450. A horizontal axis 504 represents a level of brightness or luminosity and a vertical axis 502 represents a number of pixels. Based on the brightness of the pixels of the ultrasound image data 488, the characterization circuit 450 may calculate an average (e.g., mean, geometric mean, arithmetic mean, mode, median) brightness, which may correspond to one of the image characteristic of the ultrasound image data 488. For example, the characterization circuit 450 may calculate the average brightness 508 of the ultrasound image data 488, and record the calculated average brightness 508 on the memory 490.

Additionally or alternatively, the characterization circuit 450 may determine an average contrast of the ultrasound image data 488 based from the average brightness 508. For example, the characterization circuit 450 may determine the average contrast from a ratio of the difference in brightness (e.g., subtracting a minimum brightness 512 from a maximum brightness 510) to the average brightness 508. In various other embodiments, the characterization circuit 450 may determine the average contrast of the ultrasound image data 488 by using an RMS contrast method, a Michelson contrast method, and/or the like.

At 308, one or more image display settings are adjusted corresponding to a first interface component based on the one or more image characteristics. The one or more image display settings may be adjusted by an interface adjustment circuit 451 of the controller circuit 136.

For example, the interface adjustment circuit 451 may retrieve or access the one or more image display settings corresponding to the first interface component from the memory 140, and one or more image characteristics (e.g., the average brightness 508) determined from the characterization circuit 450 stored on the memory 490. The first interface component may correspond to one of the interface components described in connection with FIG. 2, such as the drop-down menu 214. The interface adjustment circuit 451 may adjust the image display settings of the drop-down menu 214 to match, for example, a calculated mean, median, mode, and/or the like of the one or more image characteristics (e.g., the average brightness 508) of the ultrasound image data 488.

FIG. 6 is a graphical representation of a characterization histogram 600 corresponding to the brightness of the pixels of the drop-down menu 214. A horizontal axis 604 represents a level of brightness or luminosity corresponding to the image display settings and a vertical axis 602 represents a number of pixels. Based on the brightness of the pixels of the drop-down menu 214, the interface adjustment circuit 451 may calculate an average brightness 606 (e.g., mean, geometric mean, arithmetic mean, mode, median). The interface adjustment circuit 451 may adjust the image display settings of the drop-down menu 214 to match the average brightness 606 or be within a predetermined threshold of the average brightness 606 (e.g., a standard deviation above or below the average brightness 606).

For example, the interface adjustment circuit 451 may determine a brightness difference 608 between the average brightness 508 and 606 of the ultrasound image data 488 and the drop-down menu 214, respectively. Based on the brightness difference 608, the interface adjustment circuit 451 may increase the brightness of the image display settings of the drop-down menu 214 to shift the average brightness 606 to be equal and/or approximate to the average brightness 508. For example, the interface adjustment circuit 451 may increase the brightness of the image display settings for all of the pixels equally by the brightness difference 608 to shift the average brightness 608 of the drop-down menu 214. Optionally, the interface adjustment circuit 451 may store the adjusted display settings on the memory 190. It should be noted, that in other embodiments the interface adjustment circuit 451 may adjust the display settings for more than one interface component.

Additionally or alternatively, the interface adjustment circuit 451 may adjust the image display settings of the first interface component different with respect to, for example, a second interface component based on the content of the first interface component. The content of the interface component may include text and/or symbols, which may be affected by changing the image display settings.

For example, the drop-down menu 214 (e.g., the first interface component) may include text, which indicates the function(s) or operations corresponding to a selection of the drop-down menu 214. The slide bar 208 (e.g., the second interface component) may include one or more colors without having any text. The interface adjustment circuit 451 may adjust the one or more image display settings corresponding to the slide bar 208 differently than the one or more image display settings corresponding to the drop-down menu 214.

For example, the interface adjustment circuit 451 may be configured to optimize and/or have the contrast of the drop-down menu 214 be above a minimum contrast due to the text content of the drop-down menu 214. Returning to FIG. 6, the characterization histogram 600 includes two peaks 610 and 612 corresponding to two different levels of brightness 616 and 614, respectively. The peak 610, corresponding to pixels with a low brightness, having the brightness 616, may corresponding to text pixels of the drop-down menu 214. The peak 612, corresponding to pixels with a high brightness, having the brightness 612, may correspond to background pixels or pixels that surround the text of the drop-down menu 214.

When the interface adjustment circuit 451 adjusts the image display settings of the drop-down menu 214, the interface adjustment circuit 451 may only adjust the brightness of the pixels corresponding to the peak 612 to shift the average brightness 606 while increasing the contrast and/or approximately keeping the contrast of the drop-down menu 214 the same. Alternatively, when the interface adjustment circuit 451 adjust the image display settings of the drop-down menu 214, the interface adjustment circuit 451 may adjust the brightness of all the pixels equally.

In various other embodiments, the interface adjustment circuit 451 may adjust the image display settings of one or more interface components based on one or more image characteristics determined by the characterization circuit 450 from the ultrasound image 216 and the interface components displayed with the ultrasound image 216 on the display 138. For example, the characterization circuit 450 may calculate an average (e.g., geometric mean, arithmetic mean, mode, median) of the one or more image characteristics of the ultrasound image 216 and the interface components that will be displayed with the ultrasound image 216. The interface adjustment circuit 451 may adjust the image display settings of the corresponding interface components to match the average of the one or more image characteristics determined by the characterization circuit 450.

Returning to FIG. 3, at 310, the method 300 displays on a display an ultrasound image concurrently with the first interface component having adjusted one or more image display settings.

In connection with FIG. 4, the display circuit 498 accesses and obtains one or more of the ultrasound image frames and the adjusted display settings of the first interface component stored from the memory 490 or from the memory 134 and/or the memory 140 over the bus 499 to display the ultrasound image (e.g., the ultrasound image 216) concurrently with one or more interface components onto the display 138. The display circuit 498 receives user input from the user interface 142 selecting one or more ultrasound image frames to be displayed that are stored on memory (e.g., the memory 490) and/or selecting a display layout or configuration using one or more of the interface components shown on the display interface 200 for the ultrasound image frames.

The display circuit 498 of FIG. 4 may include a 2D video processor circuit 494. The 2D video processor circuit 494 may be used to combine one or more of the frames generated from the different types of ultrasound data. Successive frames of images may be stored as a cine loop (4D images) on the memory 190 or memory 140. The cine loop represents a first in, first out circular image buffer to capture image data that is displayed in real-time to the user. The user may freeze the cine loop by entering a freeze command at the user interface 142.

The display circuit 498 may include a 3D processor circuit 496. The 3D processor circuit 496 may access the memory 190 to obtain spatially consecutive groups of ultrasound image frames and to generate three-dimensional image representations thereof, such as through volume rendering or surface rendering algorithms as are known. The three-dimensional images may be generated utilizing various imaging techniques, such as ray-casting, maximum intensity pixel projection and the like.

The display circuit 498 may include a graphics circuit 497. The graphics circuit 497 may access the memory 190 to obtain groups of ultrasound image frames and the ROI data acquisition locations that have been stored or that are currently being acquired. The graphics circuit 497 may generate images that include the images of the ROI, a graphical representation positioned (e.g., overlaid) onto the images of the ROI, and the display interface 200 having one or more interface components. The graphical representation may represent an outline of a treatment space, the focal point or region of the therapy beam, a path taken by the focal region within the treatment space, a probe used during the session, the ROI data acquisition location, and the like. Graphical representations may also be used to indicate the progress of the therapy session. The graphical representations may be generated using a saved graphical image or drawing (e.g., computer graphic generated drawing), or the graphical representation may be directly drawn by the user onto the image using the display interface 200 and the user interface 142.

Additionally or alternatively, the method 300 may display on a display the first interface component without the ultrasound image 216. For example, the user selects the selectable icon 207a illustrated in FIG. 2 corresponding to a patient entry/access mode. When selected by the user, the controller circuit 136 may display one or more interface components relating to selecting and/or editing patient records (e.g., text boxes, test fields), viewing a patient history list, entering new patient information, and/or the like encompassing the activity window 210 such that the ultrasound image 216 is no longer displayed within the activity window 210. It should be noted that the image display settings of the one or more interface components displayed within the activity window 210 may still be adjusted based on the ultrasound image characteristics as described at 308 of FIG. 3.

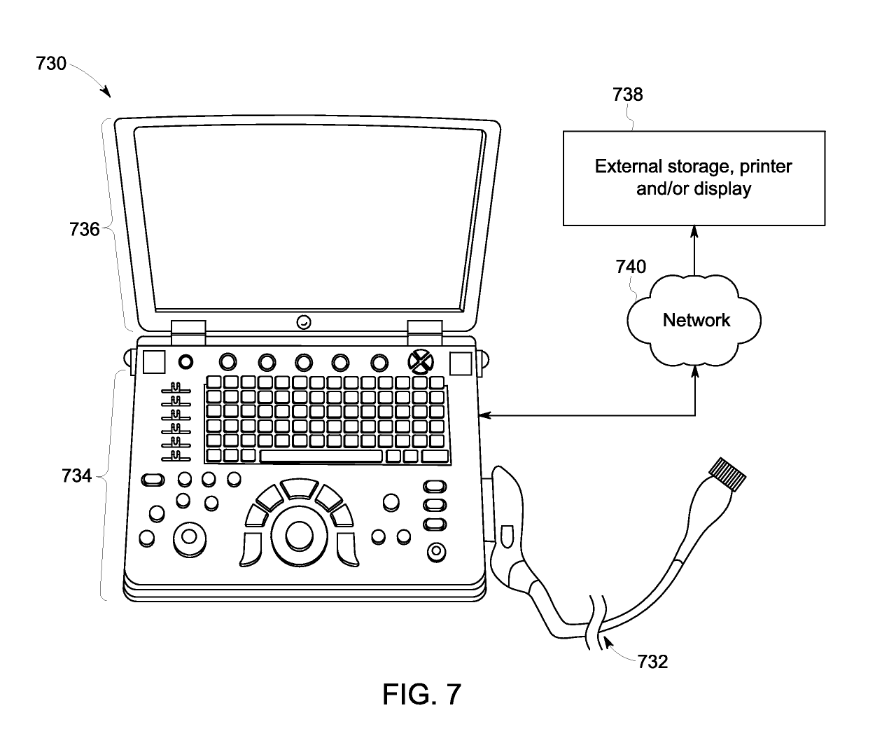

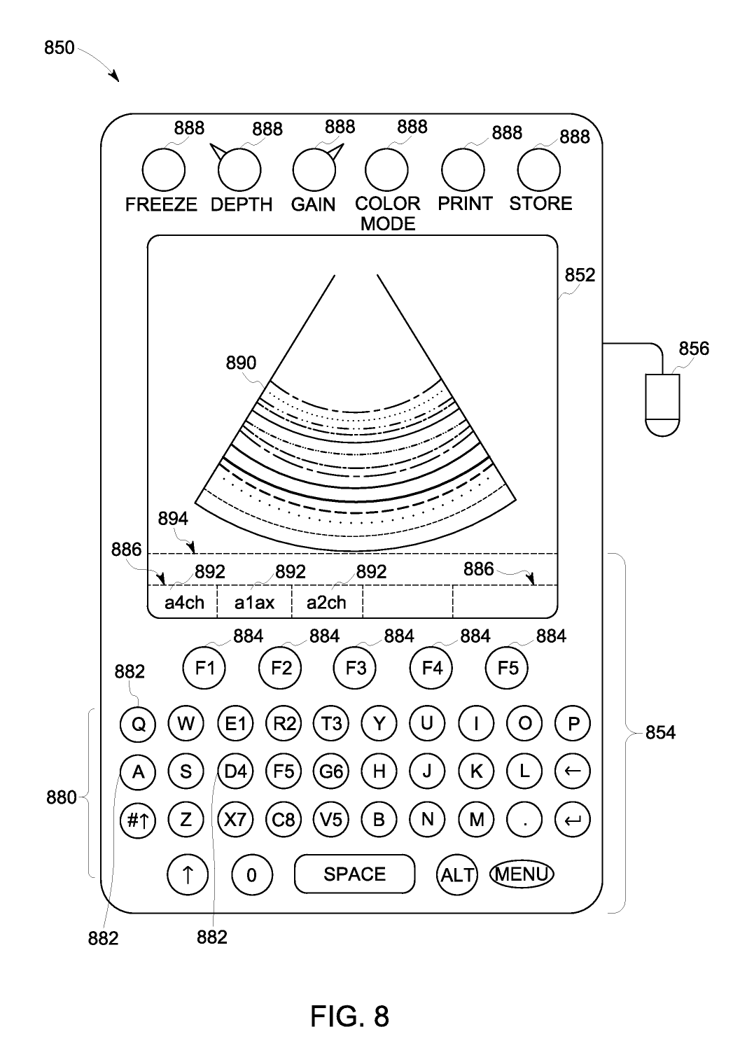

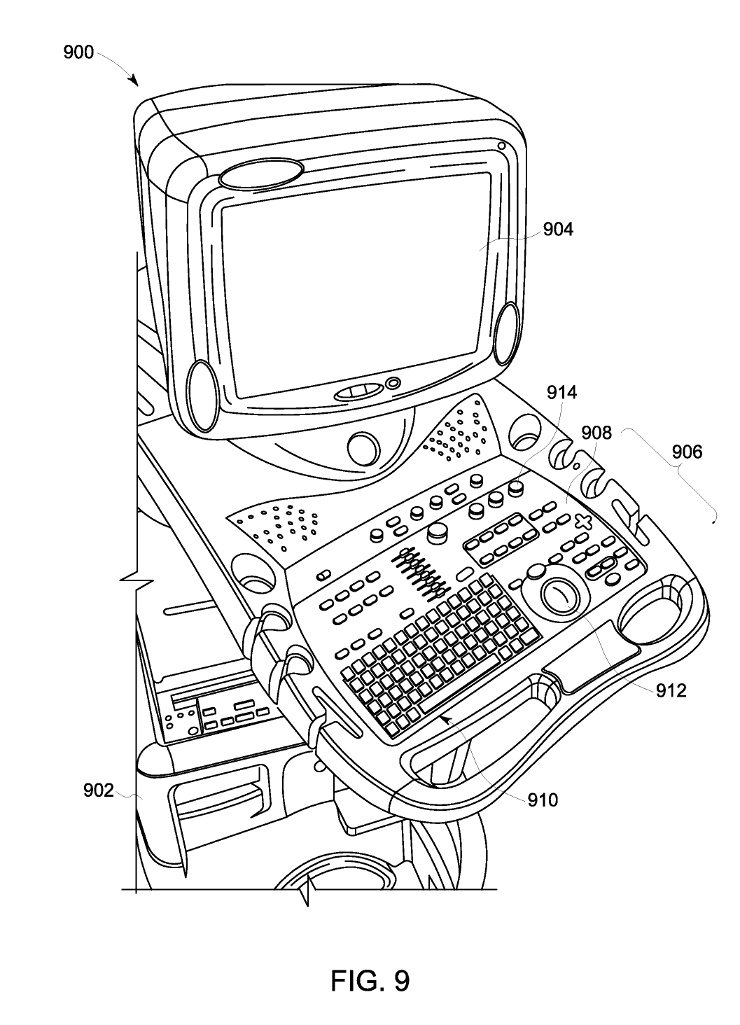

The ultrasound system 100 of FIG. 1 may be embodied in a small-sized system, such as laptop computer or pocket-sized system as well as in a larger console-type system. FIGS. 7 and 8 illustrate small-sized systems, while FIG. 9 illustrates a larger system.

FIG. 7 illustrates a 3D-capable miniaturized ultrasound system 730 having a probe 732 that may be configured to acquire 3D ultrasonic data or multi-plane ultrasonic data. For example, the probe 732 may have a 2D array of elements as discussed previously with respect to the probe. A user interface 734 (that may also include an integrated display 736) is provided to receive commands from an operator. As used herein, “miniaturized” means that the ultrasound system 730 is a handheld or hand-carried device or is configured to be carried in a person's hand, pocket, briefcase-sized case, or backpack. For example, the ultrasound system 730 may be a hand-carried device having a size of a typical laptop computer. The ultrasound system 730 is easily portable by the operator. The integrated display 736 (e.g., an internal display) is configured to display, for example, one or more medical images.

The ultrasonic data may be sent to an external device 738 via a wired or wireless network 740 (or direct connection, for example, via a serial or parallel cable or USB port). In some embodiments, the external device 738 may be a computer or a workstation having a display. Alternatively, the external device 738 may be a separate external display or a printer capable of receiving image data from the hand carried ultrasound system 730 and of displaying or printing images that may have greater resolution than the integrated display 736.

FIG. 8 illustrates a hand carried or pocket-sized ultrasound imaging system 850 wherein the display 852 and user interface 854 form a single unit. By way of example, the pocket-sized ultrasound imaging system 850 may be a pocket-sized or hand-sized ultrasound system approximately 2 inches wide, approximately 4 inches in length, and approximately 0.5 inches in depth and weighs less than 3 ounces. The pocket-sized ultrasound imaging system 850 generally includes the display 852, user interface 854, which may or may not include a keyboard-type interface and an input/output (I/O) port for connection to a scanning device, for example, an ultrasound probe 856. The display 852 may be, for example, a 320×320 pixel color LCD display (on which a medical image 890 may be displayed). A typewriter-like keyboard 880 of buttons 882 may optionally be included in the user interface 854.

Multi-function controls 884 may each be assigned functions in accordance with the mode of system operation (e.g., displaying different views). Therefore, each of the multi-function controls 884 may be configured to provide a plurality of different actions. One or more interface components, such as label display areas 886 associated with the multi-function controls 884 may be included as necessary on the display 852. The system 850 may also have additional keys and/or controls 888 for special purpose functions, which may include, but are not limited to “freeze,” “depth control,” “gain control,” “color-mode,” “print,” and “store.”

One or more of the label display areas 886 may include labels 892 to indicate the view being displayed or allow a user to select a different view of the imaged object to display. The selection of different views also may be provided through the associated multi-function control 884. The display 852 may also have one or more interface components corresponding to a textual display area 894 for displaying information relating to the displayed image view (e.g., a label associated with the displayed image).

It should be noted that the various embodiments may be implemented in connection with miniaturized or small-sized ultrasound systems having different dimensions, weights, and power consumption. For example, the pocket-sized ultrasound imaging system 850 and the miniaturized ultrasound system 830 may provide the same scanning and processing functionality as the system 100.

FIG. 9 illustrates an ultrasound imaging system 900 provided on a movable base 902. The portable ultrasound imaging system 900 may also be referred to as a cart-based system. A display 904 and user interface 906 are provided and it should be understood that the display 904 may be separate or separable from the user interface 906. The user interface 906 may optionally be a touchscreen, allowing the operator to select options by touching displayed graphics, icons, and the like.

The user interface 906 also includes control buttons 908 that may be used to control the portable ultrasound imaging system 900 as desired or needed, and/or as typically provided. The user interface 906 provides multiple interface options that the user may physically manipulate to interact with ultrasound data and other data that may be displayed, as well as to input information and set and change scanning parameters and viewing angles, etc. For example, a keyboard 910, trackball 912 and/or multi-function controls 914 may be provided.

It should be noted that although the various embodiments may be described in connection with an ultrasound system, the methods and systems are not limited to ultrasound imaging or a particular configuration thereof. The various embodiments may be implemented in connection with different types of diagnostic medical imaging systems, including, for example, x-ray imaging systems, magnetic resonance imaging (MRI) systems, computed-tomography (CT) imaging systems, positron emission tomography (PET) imaging systems, or combined imaging systems, among others.

For example, various embodiments may include a tangible and non-transitory computer readable medium comprising one or more computer software modules configured to direct one or more processors to receive medical image data acquired from a CT imaging system, an MRI system, a PET imaging system, and/or the like. The one or more computer software modules may further direct the one or more processors to determine one or more image characteristics of the medical image data as similarly described at 306 of FIG. 3, and adjust one or more image display settings corresponding to a first interface component based on the one or more image characteristics as similarly described at 308. The one or more computer software modules may further direct the one or more processors to generate a display signal for a display corresponding to a medical image (e.g., CT image, PET image, MRI image) shown concurrently with the first interface component, the first interface component displayed based on the one or more image display settings from the adjusting operation, wherein the medical image is based on the medical image data.

It should be noted that the various embodiments may be implemented in hardware, software or a combination thereof. The various embodiments and/or components, for example, the modules, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor further may include a storage device, which may be a hard disk drive or a removable storage drive such as a solid-state drive, optical disk drive, and the like. The storage device may also be other similar means for loading computer programs or other instructions into the computer or processor.

As used herein, the term “computer,” “subsystem” or “module” may include any processor-based or microprocessor-based system including systems using microcontrollers, reduced instruction set computers (RISC), ASICs, logic circuits, and any other circuit or processor capable of executing the functions described herein. The above examples are exemplary only, and are thus not intended to limit in any way the definition and/or meaning of the term “computer”.

The computer or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes of the various embodiments. The set of instructions may be in the form of a software program. The software may be in various forms such as system software or application software and which may be embodied as a tangible and non-transitory computer readable medium. Further, the software may be in the form of a collection of separate programs or modules, a program module within a larger program or a portion of a program module. The software also may include modular programming in the form of object-oriented programming. The processing of input data by the processing machine may be in response to operator commands, or in response to results of previous processing, or in response to a request made by another processing machine.

As used herein, a structure, limitation, or element that is “configured to” perform a task or operation is particularly structurally formed, constructed, or adapted in a manner corresponding to the task or operation. For purposes of clarity and the avoidance of doubt, an object that is merely capable of being modified to perform the task or operation is not “configured to” perform the task or operation as used herein. Instead, the use of “configured to” as used herein denotes structural adaptations or characteristics, and denotes structural requirements of any structure, limitation, or element that is described as being “configured to” perform the task or operation. For example, a controller circuit, processor, or computer that is “configured to” perform a task or operation may be understood as being particularly structured to perform the task or operation (e.g., having one or more programs or instructions stored thereon or used in conjunction therewith tailored or intended to perform the task or operation, and/or having an arrangement of processing circuitry tailored or intended to perform the task or operation). For the purposes of clarity and the avoidance of doubt, a general purpose computer (which may become “configured to” perform the task or operation if appropriately programmed) is not “configured to” perform a task or operation unless or until specifically programmed or structurally modified to perform the task or operation.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the various embodiments without departing from their scope. While the dimensions and types of materials described herein are intended to define the parameters of the various embodiments, they are by no means limiting and are merely exemplary. Many other embodiments will be apparent to those of skill in the art upon reviewing the above description. The scope of the various embodiments should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. §112(f) unless and until such claim limitations expressly use the phrase “means for” followed by a statement of function void of further structure.

This written description uses examples to disclose the various embodiments, including the best mode, and also to enable any person skilled in the art to practice the various embodiments, including making and using any devices or systems and performing any incorporated methods. The patentable scope of the various embodiments is defined by the claims, and may include other examples that occur to those skilled in the art. Such other examples are intended to be within the scope of the claims if the examples have structural elements that do not differ from the literal language of the claims, or the examples include equivalent structural elements with insubstantial differences from the literal language of the claims.

What is claimed is:

1. A method for controlling an ultrasound interface, comprising:
receiving ultrasound image acquisition settings for an ultrasound probe;
acquiring ultrasound image data from the ultrasound probe based on the ultrasound image acquisition settings;
determining one or more image characteristics of the ultrasound image data;
adjusting one or more image display settings corresponding to a first interface component based on the one or more image characteristics; and
displaying on a display the first interface component, the first interface component displayed based on the one or more image display settings from the adjusting operation.

2. The method of claim 1, wherein the display operation includes displaying an ultrasound image concurrently with the first interphase component, the ultrasound image is displayed based on the ultrasound image.

3. The method of claim 1, wherein the first interface component includes at least one of a drop down menu, a selectable icon, a toolbar, a menu bar, a title bar, and a window.

4. The method of claim 1, wherein the one or more image characteristics includes at least one of a hue, a contrast, a brightness, and a saturation.

5. The method of claim 1, wherein the determining operation further includes calculating a mean, a median, or a mode of the one or more image characteristics.

6. The method of claim 4, wherein the one or more image display settings are adjusted to match the calculated mean, median, or mode of the one or more image characteristics.

7. The method of claim 1, further comprising calculating a histogram representing of the one or more image characteristics of the ultrasound image data.

8. The method of claim 1, wherein the adjusting operation is further based on content of the first interface component, wherein the content includes text.

9. The method of claim 1, further comprising:
adjusting one or more image display settings corresponding to a second interface component based on the one or more image characteristics; and
displaying the second interface component on the display the ultrasound image concurrently with the first interface component.

10. The method of claim 9, wherein the one or more image display settings corresponding to the second interface component is adjusted differently than the one or more image display settings corresponding to the first interface component.

11. An ultrasound imaging system comprising:
an ultrasound probe configured to acquire ultrasound data from a region of interest;
a display; and
a controller circuit comprising at least one processor operably coupled to the ultrasound probe and the display, the controller circuit configured to:
receive ultrasound image data from the ultrasound probe;
determine one or more image characteristics of the ultrasound image data;
adjust one or more image display settings corresponding to a first interface component based on the one or more image characteristics; and
generate a display signal for the display corresponding to the first interface component, the first interface component displayed based on the one or more image display settings from the adjusting operation.

12. The ultrasound imaging system of claim 11, wherein the display signal includes an ultrasound image based on the ultrasound image data such that the ultrasound image is shown concurrently with the first interface component on the display.

13. The ultrasound imaging system of claim 11, wherein the first interface component includes at least one of a drop down menu, a selectable icon, a toolbar, a menu bar, a title bar, and a window.

14. The ultrasound imaging system of claim 11, wherein the one or more image characteristics includes at least one of a hue, a contrast, a brightness, and a saturation.

15. The ultrasound imaging system of claim 11, wherein the controller circuit is further configured to calculate a mean, a median, or a mode of one of the one or more image characteristics.

16. The ultrasound imaging system of claim 15, wherein the one or more image display settings are adjusted by the controller circuit to match the calculated mean, median, or mode of the one or more image characteristics.

17. The ultrasound imaging system of claim 11, wherein the adjusting operation by the controller circuit is further based on content of the first interface component.

18. The ultrasound imaging system of claim 11, wherein the controller circuit is further configured to:
adjusting one or more image display settings corresponding to a second interface component based on the one or more image characteristics; and
wherein the display signal includes the second interface component.

19. A tangible and non-transitory computer readable medium comprising one or more computer software modules configured to direct one or more processors to:
receive medical image data;
determine one or more image characteristics of the medical image data;
adjust one or more image display settings corresponding to a first interface component based on the one or more image characteristics; and
generate a display signal for a display corresponding to a medical image shown concurrently with the first interface component, the first interface component displayed based on the one or more image display settings from the adjusting operation, wherein the medical image is based on the medical image data.

20. The tangible and non-transitory computer readable medium of claim 17, wherein the first interface component includes at least one of a drop down menu, a selectable icon, a toolbar, a menu bar, a title bar, and a window.
